(19) United States
(12) Patent Application Publication
(10) Pub. No.: US 2004/0097502 A1
(43) Pub. Date: May 20, 2004

(54) Pyrazoles as tgf inhibitors

(76) Inventor: Francoise Jeanne Gellibert, Les Ulis (FR)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/470,862

(22) PCT Filed: Jan. 31, 2002
(86) PCT No.: PCT/GB02/00424

(30) Foreign Application Priority Data
Feb. 2, 2001 (GB) 0102670.7
Aug. 9, 2001 (GB) 0119399.4

(51) Int. Cl. A61K 31/53; A61K 31/506; A61K 31/444; C 07D 4 3/14

(52) U.S. Cl. 514/241; 514/275; 514/333; 544/209; 544/331; 546/256

(57) ABSTRACT

Therapeutically active pyrazole derivatives of formula (I) wherein R¹-R³ are as defined in the specification, processes for the preparation thereof, the use thereof in therapy, particularly in the treatment of prophylaxis of disorders characterised by overexpression of transforming growth factor β (TGF-β), and pharmaceutical compositions for use in such therapy.

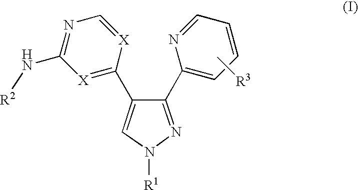

The present invention relates to novel pyrazole derivatives, processes for the preparation thereof, the use thereof in therapy, particularly in the treatment or prophylaxis of disorders characterised by overexpression of transforming growth factor β (TGF-β), and pharmaceutical compositions for use in such therapy.

TGF-β is a multi-functional cytokine which belongs to the TGF-β superfamily which includes activins/inhibins, bone morphogenetic proteins (BMPs) and TGF-βs. Three isoforms of TGF-β (TGF-β1, TGF-β2, and TGF-β3) have been identified in mammals, each of which is encoded by a distinct gene on different chromosomes (D. A. Lawrence, Eur. Cytokine. Netw., 1996, 7(3), 363). TGF-β initiates an intracellular signalling pathway which ultimately leads to the expression of genes that regulate cell cycle, control proliferative responses, or relate to extracellular matrix proteins that mediate cell adhesion, migration and intercellular communication. TGF-β has pleitropic effects including modulation of cell growth and differentiation, extracellular matrix formation, hematopolesis, and immunomodulation (Roberts and Spoon, Handbook of Experimental Pharmacology, 1990, 95, 419458).

A variety of cell surface proteins and receptors are known to transduce the signals initiated by the binding of the active TGF-β ligand to its receptors. Initiation of the TGF-β signalling pathway results from the binding of the TGF-β ligand to the extracellular domain of the type II membrane receptor (Massague, Ann. Rev. Biochem., 1998, 67, 753.). The bound type II receptor then recruits type I (Alk5) receptor into a multimeric membrane complex, whereupon, active type II receptor kinase phoshorylates and activates type I receptor kinase. The function of the type I receptor kinase is to phosphorylate a receptor-associated co-transcription factor, Smad-2 or Smad-3; thereby releasing it into the cytoplasm where it binds to Smad4. The PAI-1 gene is activated by TGF-β as a consequence of the abovementioned cellular pathway.

One approach to the treatment and/or prophylaxis of disorders characterised by the ov rexpression of TGF-β is inhibition of the TGF-β signal transduction. For example, inhibition of the TGF-β type II receptor by overexpression of a dominant negative TGF-β type II receptor has previously been shown to prevent liver fibrosis and dysfunction in rat models (Proc. Natl. Acad. Sci, 1999, 96(5), 2345), and also to prevent progression of established liver fibrosis (Hepatology, 2000, 32, 247).

Pathological overexpression of TGF-β is known to be associated with a number of undesirable effects, leading ultimately to the development of serious pathogenic conditions (G. C. Blobe et al., N. Engl. J. Med., 2000, 1350). In particular, pathological overexpression of TGF-β may cause excessive accumulation of extracellular matrix (ECM), inhibition of cell proliferation and immunosuppression. Excessive accumulation of ECM is known to lead to fibrotic diseases such as tumor fibrosis, radiation-induced fibrosis, fibrosis of the liver, kidney, lung, bowel, heart, pancreas, peritoneum or other organs. Fibrosis can lead to pathologic conditions such as cirrhosis, idiopathic pulmonary fibrosis, glomerulosclerosis and hypertrophic scars.

A number of other disease states are known to be associated with variations in the expression of genes which are controlled by TGF-β including cancer development, abnormal bone function and inflammatory disorders.

The development of compounds capable of inhibiting the TGF-β intracellular pathway is seen as a desirable way to effect prophylaxis and/or treatment of the above-mentioned conditions. Compounds capable of inhibiting the TGF-β intracellular pathway and/or the expression of TGF-β may be used in the treatment of disorders the symptoms of which often lead to the development of fibrotic conditions. For example, compounds of the present invention may be useful in treating the fibrosis associated with various liver-related conditions such as hepatitis B virus (HBV), hepatitis C virus (HCV), alcohol-induced hepatitis, haemochromatosis and primary biliary cirrhosis.

The compounds of the present invention are pyrazole derivatives. Other pyrazole compounds have previously been described for use in alternative medicinal applications. PCT Patent Applications, WO 96/03385, WO 98/52937, WO 98/52940, WO 98/52941 and WO 00/31063 (Searle & Co) disclose a series of substituted pyrazole compounds and their use in the treatment of p38 kinase mediated disorders. In particular the compounds described are useful in the treatment of inflammation, inflammatory bowel disease, multiple sclerosis and asthma. European Patent Application No. 0 846 687 (Lilly & Co) describes novel substituted pyrazoles useful for the inhibition of sPLA₂ mediated release of fatty acids. Such compounds are useful in the treatment of conditions such as septic shock. EP 0 846 686 (Pfizer Ltd) discloses a series of condensed pyrazole derivatives which act as inhibitors of both Interleukin-1 (IL-1) and tumor necrosis factor (TNF). Such compounds are useful in the treatment of IL-1 and TNF mediated diseases such as chronic inflammatory diseases, specific autoimmune disaease and sepsis-induced organ injury. None of the aforementioned patent applications describe the pyrazole compounds of the present invention.

PCT Patent Application WO 00/12947 (Scios Inc.) describes the use of a series of quinazoline derivatives for treating various disorders associated with enhanced activity of kinase p38-α and/or TGF-β. The compounds described therein have been shown to inhibit the activities of both proteins and are therefore particularly useful for the treatment of conditions in which an,enhanced activity towards both p38-α and TGF-β is required.

It has now been discovered that certain substituted pyrazole compounds, as described below, are useful in the treatment or prophylaxis of disorders characterised by the overexpression of TGF-β. In particular, compounds of the present invention are TGF-β inhibitors which act at the TGF-β type I (Alk5) receptor.

According to one aspect of the present invention, we provide compounds of formula (I),

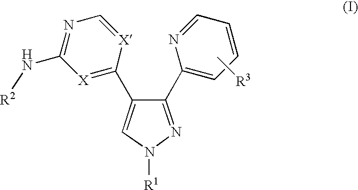

wherein,

R¹ is selected from H, C₁₋₄ alkyl or CH₂CONR⁴R⁵;

R² is selected from —(CH₂)ₙ-phenyl, —(CH₂)ₙ-heterocyclyl, —(CH₂)ₙ-heteroaryl, each of which may be further substituted by one or more substituents, which may be the same or different, selected from halo (such as fluoro, chloro, bromo), —CN, —CF₃, —OH, —OCF₃, C₁₋₄ alkyl or C₁₋₄ alkoxy, —NO₂, —NH₂, —NR⁴R⁶, —CONR⁴R⁶, —NHCOR⁴, —SO₂R⁴, —SO₂NHR⁴, —O(CH₂)ₙNR⁴R⁶;

R³ is selected from H, halo (such as fluoro, chloro, bromo), —CN, —CF₃, C₁₋₄ alkyl or C₁₋₄ alkoxy;

R⁴ is selected from H or C₁₋₄ alkyl;

R⁵ is C₁₋₄ alkyl;

R⁶ is selected from heterocyclyl or heteroaryl;

or R⁴R⁶ together with the nitrogen atom to which they are attached form a 3, 4, 5, 6 or 7 membered saturated or unsaturated ring which may additionally contain one or more heteroatoms selected from N, S or O, and wherein the ring may be further substituted by one or more substitutents selected from halo (such as fluoro, chloro, bromo), —CN, —CF₃, —OH, —OCF₃, C₁₋₄ alkyl and C₁₋₄ alkoxy;

n is 0, 1, 2, 3, 4 or 5;

X and X′, which may be the same or different, are each selected from CH or N, provided that X and X′ are not both N;

and salts and solvates thereof (hereinafter “compounds of the invention”).

As used herein the term “alkyl” as a group or part of a group refers to a straight or branched chain saturated aliphatic hydrocarbon radical containing the specified number(s) of carbon atoms. Such alkyl groups in particular include methyl, ethyl, n-propyl, iso-propyl, n-butyl, sec-butyl, tert-butyl, pentyl and hexyl.

The term “alkoxy” as a group or part of a group refers to an alkyl ether radical, wherein the term “alkyl” is defined above. Such alkoxy groups in particular include methoxy, ethoxy, n-propoxy, iso-propoxy, n-butoxy, iso-butoxy, sec-butoxy and tert-butoxy.

The term “heterocyclyl ” as a group or part of a group refers to a stable saturated or partially saturated (i.e. non-aromatic) 3 to 6 membered monocyclic ring containing one or more hetero atoms independently selected from nitrogen, oxygen and sulfur, optionally substituted with one or more substituents, which may be the same or different, selected from halo (such as fluoro, chloro, bromo), —CN, —CF₃, —OH, —OCF₃, C₁₋₄ alkyl and C₁₋₄ alkoxy.

The term “heteroaryl” as a group or part of a group refers to a stable heterocyclic aromatic 6 to 14 membered monocyclic ring containing one or more hetero atoms independently selected from nitrogen, oxygen and sulfur, optionally substituted with one or more substituents, which may be the same or different, selected from halo (such as fluoro, chloro, bromo), —CN, —CF₃, —OH, —OCF₃, C₁₋₄ alkyl and C₁₋₄ alkoxy.

The present invention also covers the physiologically acceptable salts of the compounds of formula (I). Suitable physiologically acceptable salts of the compounds of formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of formula (I), for example hydrates.

Compounds of formula (I) wherein R¹ is H may exist in tautomeric forms. The individual tautomers and mixtures thereof are included within the scope of the present invention.

Preferably, R¹ is H or C₁₋₄ alkyl, more preferably R¹ is H.

Preferably, n is 0 or 1.

Preferably, R² is —(CH₂)ₙ-phenyl or —(CH₂)ₙ-heterocyclyl. More preferably, R² is —(CH₂)ₙ-phenyl or —(CH₂)ₙ-heterocyclyl substituted by one or more substituents selected from halo (such as fluoro, chloro, bromo), —CN, —CF₃, —OH, —OCF₃, C₁₋₄ alkyl, C₁₋₄ alkoxy, —CONR⁴R⁶, —SO₂R⁴ or —O(CH₂)ₙNR⁴R⁶. Most preferably, n is 0 and R² is phenyl substituted by one or more substituents selected from halo (such as fluoro, chloro, bromo), —CN, —CF₃, —OH, —OCF₃, C₁₋₄ alkyl, C₁₋₄ alkoxy, —CONR⁴R⁶, —SO₂R⁴ or —O(CH₂)ₙNR⁴R⁶ and n is 1; or n is 1 and R² is furanyl.

Preferably, R³ is located at the C(3) or C(6) position of the pyridine ring and is selected from H, halo (such as fluoro, chloro, bromo), —CN, —CF₃, C₁₋₄ alkyl or C₁₋₄ alkoxy. More preferably, R³ is H or altematively C₁₋₄ alkyl. Most preferably, R³ is H.

Preferably R⁴ and R⁶ together with the nitrogen atom to which they are attached represent pyrroline, pyrrolidine, imidazole, N-substituted imidazole, imidazoline, imidazolidine, pyrazole, pyrazoline, pyrazolidine, piperidine, morpholine, thiomorpholine, piperazine, N-substituted piperazine.

More preferably R⁴ and R⁶ together with the nitrogen atom to which they are attached represent pyrrolidine, piperidine, morpholine, piperazine, N-substituted piperazine (preferably N-methyl-piperazine), imidazole, or N-substituted imidazole (preferably N-methyl-imidazole).

It will be appreciated that the present invention Is intended to include compounds having any combination of the preferred groups as listed hereinbefore.

Compounds of formula (I) which are of special interest as agents useful in the treatment or prophylaxis of disorders characterised by the overexpression of TGF-β are:

(4-Chlorophenyl)-[4-(3-pyridin-2-yl)-1H-pyrazol-4-yl)-pyridin-2-yl]-amine;

Phenyl-[4-(3-pyridin-2-yl)-1H-pyrazol-4-yl)pyridin-2-yl]-amine;

(4-Fluoro-phenyl)-[4-(3-pyridin-2-yl)-1H-pyrazol-4-yl)-pyridin-2-yl]-amine;

Furan-2-ylmethyl-[4-(3-pyridin-2-yl)-1H-pyrazol-4-yl)-pyridin-2-yl]-amine;

(3-Methanesulfonyl-phenyl)-[4-(3-pyridin-2-yl)-1H-pyrazol-4-ylypyridin-2-yl]-amine;

3-[4-(3-Pyridin-2-yl)-1H-pyrazol-4-yl)-pyridin-2-yl-amino]-benzonitrile;

2-Methoxy-4-[4-(3-pyridin-2-yl)-1H-pyrazol-4-yl)-pyridin-2-yl-amino]-benzonitrile;

[4-(3-Pyridin-2-yl)-1H-pyrazol-4-yl)-pyridin-2-yl]-[3-(2-piperidin-1-yl-ethoxy)-phenyl]-amine;

[4-(3-Pyridin-2-yl)-1H-pyrazol-4-yl)-pyridin-2-yl]-[3-(2-pyrrolidin-1-yl-ethoxy)-phenyl]-amine;

(3-Chloro-phenyl)-[4-(3-pyridin-2-yl)-1H-pyrazol-4-yl)-pyridin-2-yl]-amine;

[4-(3-Pyridin-2-yl-1H-pyrazol-4-yl)-pyridin-2-yl]-m-tolyl-amine;

[4-(3-Pyridin-2-yl-1H-pyrazol-4-yl)-pyridin-2-yl]-[4-(2-pyrrolidin-1-yl-ethoxy)-phenyl]-amine;

(3,4-Dimethoxy-phenyl)-[4-(3-pyridin-2-yl-1H-pyrazol-4-yl)-pyridin-2-yl]-amine;

(4-Chloro-3-methyl-phenyl)-[4-(3-pyridin-2-yl-1H-pyrazol-4-yl)-pyridin-2-yl]-amine;

2-Chloro-5-[4-(3-pyridin-2-yl-1H-pyrazol-4-yl)-pyridin-2-ylamino]-benzonitrile;

4-[4-(3-Pyridin-2-yl-1H-pyrazol-4-yl)-pyridin-2-ylamino]-phenol;

(4-Methoxy-phenyl)-[4-(3-pyridin-2-yl-1H-pyrazol-4-yl)-pyridin-2-yl]-amine;

[4-(3-Pyridin-2-yl-1H-pyrazol-4-yl)-pyridin-2-yl]-p-tolyl-amine;

[4-(3-Pyridin-2-yl-1H-pyrazol-4-yl)-pyridin-2-yl]-(3-trifluoromethoxy-phenyl)-amine;

(3,4-Dimethyl-phenyl)-[4-(3-pyridin-2-yl-1H-pyrazol-4-yl)pyridin-2-yl]-amine;

[4-(3-Pyridin-2-yl-1H-pyrazol-4-yl)-pyridin-2-yl]-(4-trifluoromethyl-phenyl)-amine;

(3-Isopropoxy-phenyl)-[4-(3-pyridin-2-yl-1H-pyrazol-4-yl)-pyridin-2-yl]-amine;

(4-Methanesulfonyl-phenyl)-[4-(3-pyridin-2-yl-1H-pyrazol-4-yl)-pyridin-2-yl]-amine;

2-Methoxy-4-{4-[3-(6-methyl-pyridin-2-yl)-1H-pyrazol-4-yl]-pyridin-2-ylamino}-benzonitrile;

Morpholin-4-yl-{4-[4-(3-Pyridin-2-yl-1H-pyrazol-4-yl)-pyridin-2-ylamino]-phenyl}methanone;

(4-Methyl-piperazin-1-yl)-{4-[4-(3-pyridin-2-yl-1H-pyrazol-4-yl)-pyridin-2-ylamino]-phenyl}-methanone;

[4-(2-Piperidin-1-yl-ethoxy)-phenyl]-[4-(3-pyridin-2-yl-1H-pyrazol-4-yl)-pyridin-2-yl]-amine; and

[3-(1-Methyl-1H-imidazol-2-ylmethoxy)-phenyl]-[4-(3-pyridin-2-yl-1H-pyrazol-4-yl)-pyridin-2-yl]-amine;

and salts and solvates thereof.

Compounds of formula (I) and salts and solvates thereof may be prepared by the methodology described hereinafter, constituting a further aspect of this invention.

Compounds of formula (I) may conveniently be prepared according to the general methodologies in Schemes 1, 2 and 3 below:

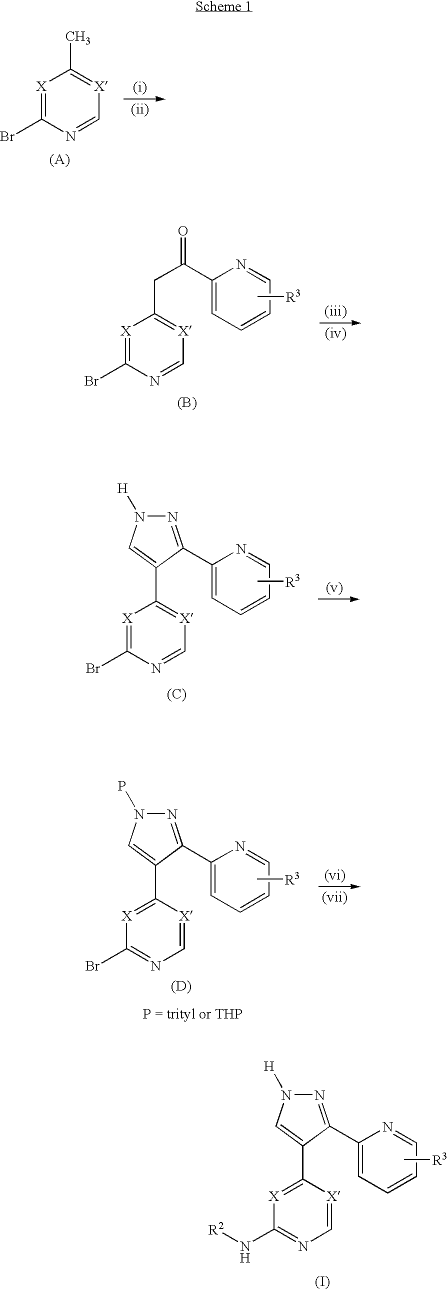

Reagents and conditions (preferred): (i) NaHMDS, THF, −50° C.; (ii) R³(C₅H₃N)CO₂Et, THF, −50° C.; (iii) DMF.DMA,AcOH, DMF, r.t.; (iv) NH₂NH₂.H₂O, DMF, r.t.; (v) trityl chloride, K₂CO₃, acetone, reflux or DHP, PPTS, DCE, r.t.; (vi) R²NH₂, Pd₂(dba)₃, binap, NaOBu^{t}, toluene, 80° C.; (vii) HCl (1N), MeOH, reflux; or TFA, CH₂Cl₂, r.t.

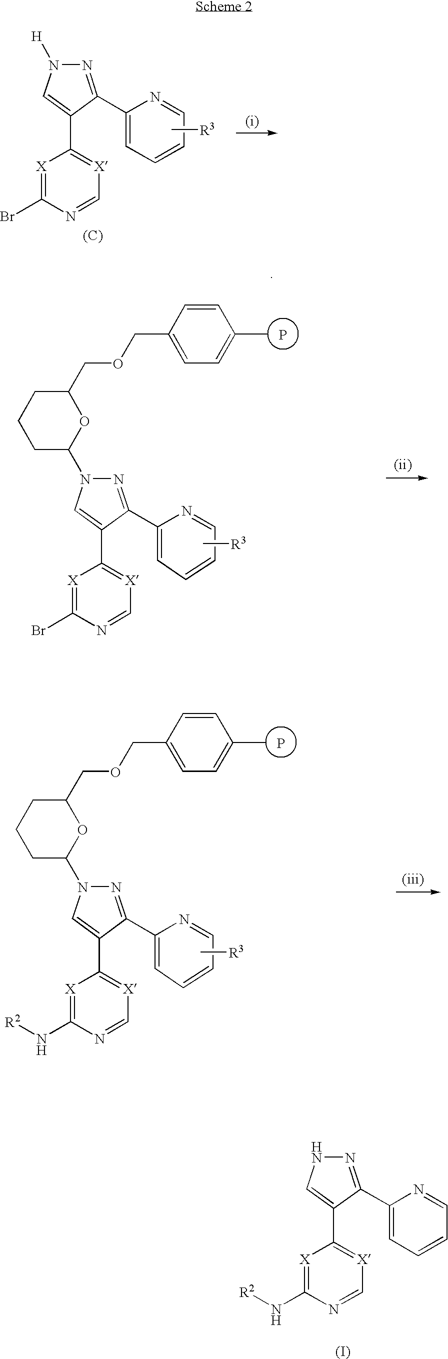

Reagents and conditions (preferred): (i) polystyrene DHP resin, PPTS, CH₂Cl₂, 75° C.; (ii) R²NH₂, Pd₂(dba)₃, binap, NaOBu^{t}, toluene/EtOH, 90° C.; (iii) 10%TFA/CH₂Cl₂, r.t.

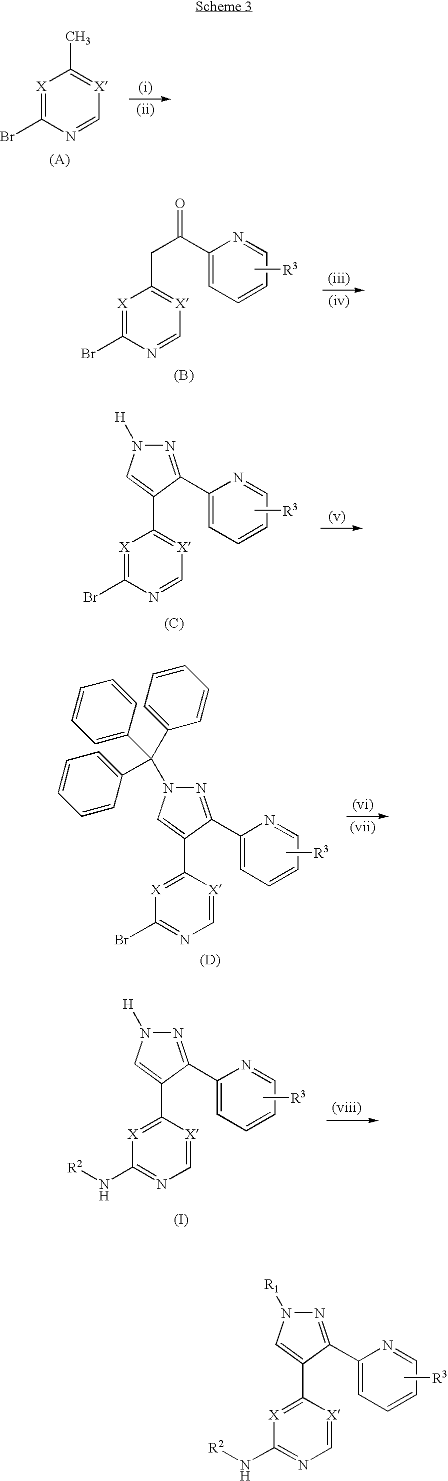

Reagents and conditions (preferred): (i) NaHMDS, THF, −50° C.; (ii) R³(C₅H₃N)CO₂Et, THF, −50° C.; (iii) DMF.DMA, AcOH, DMF, r.t.; (iv) NH₂NH₂.H₂O, DMF, r.t.; (v) trityl chloride, K₂CO₃ or DHP, PPTS, DCE, r.t.; (vi) R²NH₂, Pd₂(dba)₃, binap, toluene, NaOBu^{t}, 80° C.; (vii) HCl (1N), MeOH, reflux; or TFA, CH₂Cl₂, r.t.; (viii) R¹Y, K₂CO₃, DMF, r.t.

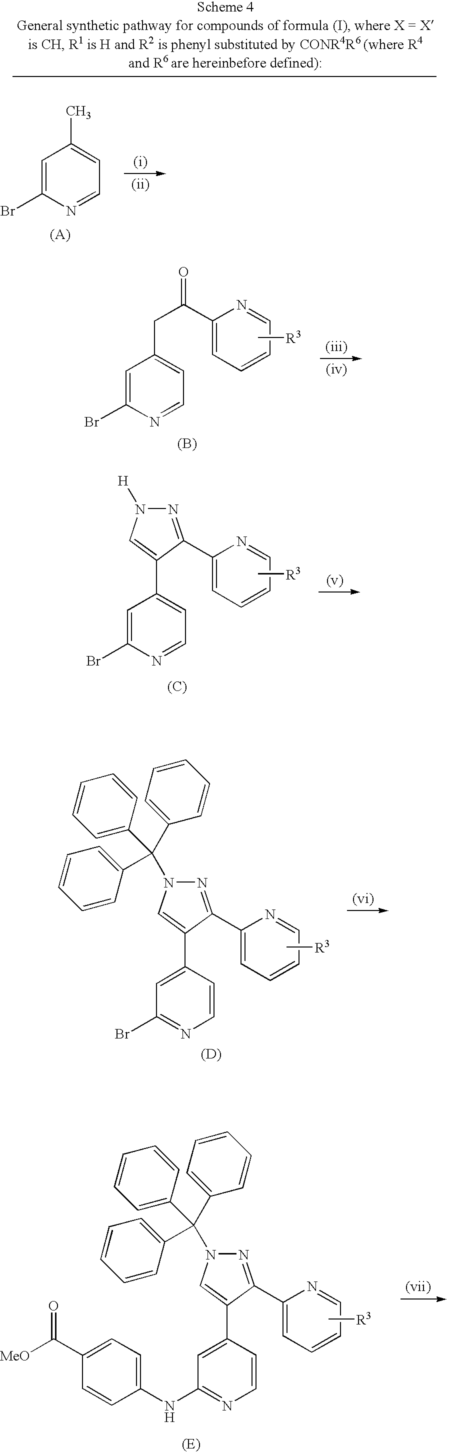

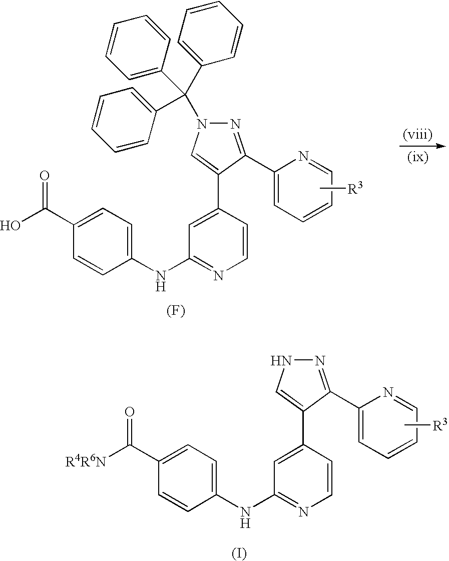

Reagents and conditions (preferred): (i) NaHMDS, THF, −50° C.; (ii) R³(C₅H₃N)CO₂Et, THF, −50° C.; (iii) DMF.DMA, AcOH, DMF, r.t.; (iv) NH₂NH₂.H₂O, DMF, r.t.; (v) trityl chloride, K₂CO₃, acetone, reflux; (vi) 4-amino-benzoic acid, methyleester, Pd₂(dba)₃, binap, NaOBu^{t}, toluene, 80° C.; (vii) NaOH (1N), MeOH, reflux; (viii) R⁴R⁶NH, HOBT, EDCl, CH₂Cl₂, r.t; (ix) HCl (1N), MeOH, reflux.

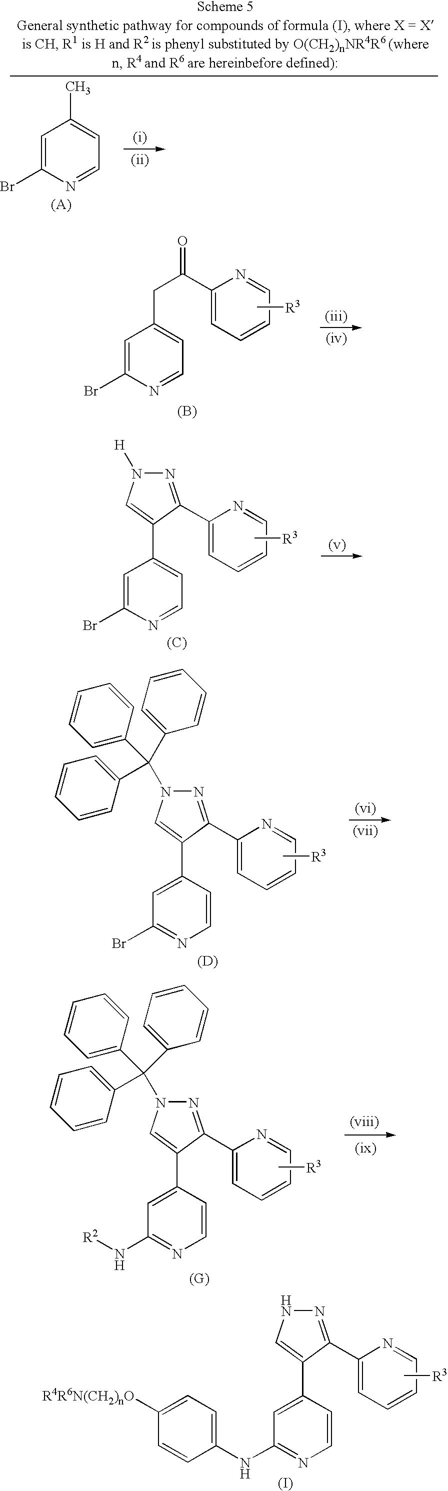

Reagents and conditions (preferred): (i) NaHMDS, THF, −50° C.; (ii) R³(C₅H₃N)CO₂Et, THF, −50° C.; (iii) DMF.DMA, AcOH, DMF, r.t.; (iv) NH₂NH₂.H₂O, DMF, r.t.; (v) trityl chloride, K₂CO₃, acetone, reflux; (vi) 4-benzyloxy-phenylamine hydrochloride, Pd₂(dba)₃, binap, NaOBu^{t}, toluene, 80° C.; (vii) Pd/C 10%, EtOH, 40° C.; (viii) Cs₂CO₃, acetone, R⁴R⁶NH, reflux; (ix) HCl (1N), MeOH, reflux.

List of Abbreviations

| | |
|---|---|
| AcOH | acetic acid |
| Binap | 2,2′-Bis(diphenylphosphino)-1,1′-binaphthyl |
| DMF.DMA | Dimethylformamide dimethylacetal |
| DMF | Dimethylformamide |
| DHP | Dihydropyrane |
| DCE | Dichioroethane |
| DIEA | Diisopropylethylamine |
| EDCL | 1-(3-Dimethylaminopropyi)-3-ethylcarbodiimide |
| | hydrochloride |
| HOBT | 1-Hydroxybenzotriazole hydrate |
| NaHMDS | Sodium bis(tnmethylsilyl)amide |
| MeCN | Acetonitrile |
| PPTS | Pyridinium p-toluenesulfonate |
| Pd₂(dba)₃ | Tris(dibenzylideneacetone)dipalladium(0) |
| TFA | Trifluoroacetic acid |
| THF | Tetrahydrofuran |
| THP | Tetrahydropyran |

A general process according to the invention for preparing a compound of formula (I) wherein R¹ is H, comprises:

(i) Addition of a suitable base, such as sodium bis(trimethylsilyl)amide or potassium bis(trimethylsilyl)amide to a substituted pyridine of formula (A), preferably in the temperature range 0 to −80° C., more preferably in the temperature range −30 to −60° C., most preferably at −50° C., in the presence of a suitable solvent such as THF;

(ii) Addition of a suitable monosubstituted pyridyl ester, R³(C₅H₅N)CO₂Et (wherein R³ is hereinbefore defined) to the reaction mixture, preferably in the temperature range 0 to −80° C., more preferably in the temperature range −30 to −60° C., most preferably at −50° C., in the presence of a suitable solvent such as THF;

(iii) Addition of dimethylformamide dimethylacetal to the resulting ketone (B), preferably in the temperature range 0 to 75° C., more preferably in the temperature range 20 to 60° C., most preferably at room temperature, in the presence of AcOH and a suitable solvent such as DMF; and

(iv) Addition of hydrazine monohydrate, NH₂NH₂.H₂O, preferably in the temperature range 0 to 75° C., more preferably in the temperature range 20 to 60° C., most preferably at room temperature, in the presence of a suitable solvent such as DMF.

It will be appreciated by a person skilled in the art that N-alkylation according to step (viii), Scheme 3 with an N-alkylating agent R¹Y (where Y is a suitable leaving group such as Cl or Br), may result in the formation of structural isomers of a compound of formula (I). Such isomers are afforded by N-alkylation of the tautomeric forms of a compound of formula (I) where R¹ is H, mentioned hereinbefore. The individual isomers and mixtures thereof are included within the scope of the present invention.

Substituted pyridine compounds of formula (A) may be prepared by processes analogous to those known in the art (e.g. Osuch et al., J. Org. Chem., 1957, 22, 939). Examples of such preparative procedures are provided in the specific examples hereinafter.

2-Bromo-4-methylpyrimidine can be prepared from 2-amino4-methylpyrimidine (commercial) as described in the literature: Mukkala, Veli-Matti; Sund, Christian; Kwiatkowski, Marek; Pasanen, Paavo; Hoegberg, Maria; et al.; Helv.Chim.Acta; 75; 5; 1992; 1621-1632.

Monosubstituted pyridyl esters, R³(C₅H₃N)CO₂Et (where R³ is as hereinbefore defined) as described in step (ii) of Schemes 1 and 3 above may be prepared by processes analogous to those known in the art. For example, where R³═C(6)-OMe, Finger et al., J. Org. Chem., 1962, 27, 3965; where R³═C(3)-OMe, Dejardin et al., Bull. Chim. Soc. Fr., 1979, 289; where R³═C(5)-Br, Chambers and Marfat, Synth. Commun., 1997, 27(3), 515; and where R³═C(4)-CN, Heinisch and Lotsch, Heterocycles, 1987, 26(3), 731.

N-alkylation reactions as described in step (viii), Scheme 3 may be performed according to processes analogous to those known in the art (e.g. R. Fusco, Pyrazoles, Chapter 4, p.71, The Chemistry of Heterocyclic Compounds, A. Weissberger (ed), Vol. 22, Intersciences, New York, 1967 and Elguero, Pyrazoles and their Benzo derivatives, p.222, Comprehensive Heterocycles Chemistry, A. R. Katrisky, C. W. Rees and K. T. Potts (eds), Vol. 5, Pergamon Press, Oxford, 1984).

The compounds of the present invention have been found to inhibit phosphorylation of the Smad-2 or Smad-3 proteins by inhibition of the TGF-β type 1 (Alk5) receptor.

Accordingly, the compounds of the invention have been tested in the assays described herein and have been found to be of potential therapeutic benefit in the treatment and prophylaxis of disorders characterised by the overexpression of TGF-β.

Thus there is provided a compound of formula (I) or a physiologically acceptable salt or solvate thereof for use as a medicament in human or veterinary medicine, particularly in the treatment or prophylaxis of disorders characterised by the overexpression of TGF-β.

It will be appreciated that references herein to treatment extend to prophylaxis as well as the treatment of established conditions. It will further be appreciated that references herein to treatment or prophylaxis of disorders characterised by the overexpression of TGF-β, shall include the treatment or prophylaxis of TGF-β associated disease such as fibrosis, especially liver and kidney fibrosis, cancer development, abnormal bone function and inflammatory disorders.

Other pathological conditions which may be treated in accordance with the invention have been discussed in the introduction hereinbefore. The compounds of the present invention are particularly suited to the treatment of fibrosis and related conditions.

Compounds of the present invention may be administered in combination with other therapeutic agents, for example antiviral agents for liver diseases, and in combination with ACE inhibitors or Angiotensin II receptor antagonists for kidney diseases.

According to another aspect of the invention, there is provided the use of a compound of formula (I) or a physiologically acceptable salt or solvate thereof for the manufacture of a medicament for the treatment and/or prophylaxis of disorders characterised by the overexpression of TGF-β, particularly fibrosis.

In a further aspect there is provided a method for the treatment of a human or animal subject with a disorder characterised by the overexpression of TGF-β, particularly fibrosis, which method comprises administering to said human or animal subject an effective amount of a compound of formula (I) or a physiologically acceptable salt or solvate thereof.

Compounds of the invention may be formulated for administration in any convenient way, and the invention therefore also includes within its scope pharmaceutical compositions for use in therapy, comprising a compound of formula (I) or a physiologically acceptable salt or solvate thereof in admixture with one or more physiologically acceptable diluents or carriers.

There is also provided according to the invention a process for preparation of such a pharmaceutical composition which comprises mixing the ingredients.

Compounds of the invention may, for example, be formulated for oral, buccal, parenteral, topical or rectal administration.

Tablets and capsules for oral administration may contain conventional excipients such as binding agents, for example syrup, acacia, gelatin, sorbitol, tragacanth, mucilage of starch or polyvinyl pyrrolidone; fillers, for example, lactose, microcrystalline cellulose, sugar, maize-starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art.

Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavouring, colouring and/or sweetening agents (e.g. mannitol) as appropriate.

For buccal administration the compositions may take the form of tablets or lozenges formulated in conventional manner.

The compounds may also be formulated as suppositories, e.g. containing conventional suppository bases such as cocoa butter or other glycerides.

Compounds of the invention may also be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or toxicity adjusting agents. Alternatively, the active ingredient may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

By topical administration as used herein, we include administration by insufflation and inhalation. Examples of various types of preparation for topical administration include ointments, creams, lotions, powders, pessaries, sprays, aerosols, capsules or cartridges for use in an inhaler or insufflator or drops (e.g. eye or nose drops).

Ointments and creams may, for example, be formulated with an aqueous or oily base with the addition of suitable thickening and/or gelling agents and/or solvents. Such bases may thus, for example, include water and/or an oil such as liquid paraffin or a vegetable oil such as arachis oil or castor oil or a solvent such as a polyethylene glycol. Thickening agents which may be used include soft paraffin, aluminium stearate, cetostearyl alcohol, polyethylene glycols, microcrystalline wax and beeswax.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents or suspending agents.

Spray compositions may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluorethane, carbon dioxide or other suitable gas.

Capsules and cartridges for use in an inhaler or insufflator, of for example gelatin, may be formulated containing a powder mix of a compound of the invention and a suitable powder base such as lactose or starch.

Compounds of the invention may conveniently be administered in amounts of, for example, 0.01 to 100 mg/kg body weight, suitably 0.05 to 25 mg/kg body weight orally, one or more times a day. The precise dose will of course depend on the age and condition of the patient, the particular route of administration chosen, and is entirely within the discretion of the administering physician.

The following non-limiting Examples illustrate the present invention.

Intermediates

Intermediate B1:

1-Pyridin-2-yl-2-[2-bromo-pyridin-4-yl]-ethanone

To a solution of 2-bromo4-methyl-pyridine (27 g) in dry THF (270 ml) was added ethyl picolinate (28.5 g), the resulting mixture was cooled to −78° C. under argon. A solution of sodium bis-(trimethylsilyl)amid 1M in THF (345 ml) was added dropwise at −78° C. and after completion the reaction mixture was allowed to warm to room temperature and stirred overnight. The solvent was evaporated under reduced pressure and the solid residue was triturated with diethyl ether, filtered and washed with diethyl ether. The solid was then diluted with saturated NH₄Cl solution and the aqueous phase was extracted with ethyl acetate. The organic layer was dried over Na₂SO₄ and concentrated. The resulting orange powder was washed with pentane to give the title compound as a yellow solid (33.97 g).

m.p: 111.2° C.

¹H NMR (CDCl₃): δ8.56 (d, 1H); 8.12 (d, 1H); 7.9 (d, 1H); 7.7 (td, 1H);7.39-7.34 (m, 1H); 7.33 (s, 1H); 7.06 (d, 1H); 4.37 (s, 2H).

Intermediate B2:

2-[2-Bromo-pyridin-4-yl]-1-(6-methyl-pyridin-2-yl)-ethanone

2-Bromo-4-methyl-pyridine (5 g, 29 mmol) and methyl-6-methylpicolinate (1.1 eq, 4.82 g, 32 mmole) were reacted as described for intermediate B1 to afford the title compound as a yellow solid (5.84 g, 70%)

MS m/z: 292 (MH+)

Intermediate C1:

2-Bromo-4-(3-pyridin-2-yl-1H-pyrazol-4-yl)pyridine

A solution of 1-pyridin-2-yl-2-[2-bromo-pyridin-4-y]-ethanone (69.68 g, 0.251 mol) in dry DMF (13.5 ml) under nitrogen was treated with glacial acetic acid (2.4 eq, 35 ml, 0.61 mol) over 2 min. DMF.DMA (1.5 eq., 50 ml, 0.376 mol) was added dropwise and the mixture was stirred at room temperature under nitrogen for 78 min. Hydrazine monohydrate (7.5 eq, 91 ml, 1.876 mol) was added dropwise, the resulting mixture was heated at 50° C. for 2 h and then allowed to cool and stirred at room temperature overnight. The reaction mixture was poured into water (4 l) and extracted with ethyl acetate (1 l). The organic extracts were dried over Na₂SO₄ and filtered. Reduction in vacuo afforded a brown oil which was crystallised from acetonitrile to yield the title compound as cream solid (45.8 g, 60.5%).

¹H NMR (CDCl₃): δ8.62 (d, 1H); 8.28 (d, 1H); 7.68 (s, 1H); 7.62 (td, 1H); 7.51 (s, 1H); 7.39 (d, 1H); 7.26-7.22 (m, 2H).

Intermediate C2:

2-bromo-4-(3-(6-methyl-pyridin-2-yl)-1H-pyrazol-4-yl)pyridine

2-[2-bromo-pyridin-4-yl]-1-(6-methyl-pyridin-2-yl)-ethanone (5.84 g, 20 mmol) was reacted as described for intermediate C1 to afford, the title compound as a yellow solid (3.07 g, 49%)

MS m/z: 315 (MH+)

Intermediate D1:

2-Bromo-4-(3-pyridin-2-yl-1-trityl-1H-pyrazol-4-yl)pyridine

To a solution of 2-bromo-4-(3-pyridin-2-yl-1H-pyrazol-4-yl)pyridine (10.6 g) in acetone (200 ml) were added potassium carbonate (3 eq, 14.6 g) and trityl chloride (1.5 eq, 14.8 g). The reaction was heated to reflux and stirred for a further 24 h. After filtration, the filtrate was concentrated and then partitioned between CH₂Cl₂ and H₂O. The organic phase was dried over Na₂SO₄ and concentrated. The resulting crude material was purified by flash chromatography eluting with CH₂Cl₂/MeOH (98:2) to give the title compound as a white solid (16.3 g, 85.3%), which contains the 2-trityl isomer as a minor component.

m.p 202° C.

MS m/z 544 (MH+)

Intermediate D2:

2-Bromo-4-[3-(6-methyl-pyridin-2-yl)-1-trityl-1H-pyrazol-4-yl]pyridine

2-Bromo-4-(3-(6-methyl-pyridin-2-yl)-1H-pyrazol-4-yl)pyridine (3.07 g , 9.8 mmol) and trityl chloride (1.5 eq, 4.1 g, 14.7 mmol) were reacted as described for Intermediate D1 to afford the title compound as the major isomer of a mixture of the two isomers, as a light yellow solid (4.9 g, 90%).

MS m/z: 558 (MH+)

Intermediate E:

4-[4-(3-Pyridin-2-yl-1-trityl-1H-pyrazol-4-yl)-pyridin-2-ylamino]-benzoic acid methyl ester

To a suspension of Intermediate D (5.5 g) in toluene (200 ml) were added Pd₂(dba)₃ (190 mg, 2 mol %), binap (257 mg, 4 mol %), 4-Amino-benzoic acid methyl ester (2.03 g, 1.3 eq) and sodium tert-butoxide (1.4 g, 1.4 eq). The reaction mixture was stirred at 80° C. overnight. The mixture was poured in cold water and extracted with AcOEt. The organic layer was washed with saturated NaCl solution, dried over Na₂SO₄, filtered, evaporated to dryness and chromatographed [CH₂Cl₂/EtOH 98:2] to afford the title compound which contains the 2-trityl isomer as a minor component (3.72 g) as an orange oil.

Calcd. Mass for C₄₀H₃₁N₅O₂: (M+1)⁺:614.2556. Found (H.R.M.S): 614.2536.

Intermediate F:

4-[4-(3-Pyridin-2-yl-1-trityl-1H-pyrazol-4-yl)-pyridin-2-ylamino]-benzoic acid

To a solution of 4-[4-(3-pyridin-2-yl-1-trityl-1H-pyrazol-4-yl)-pyridin-2-ylamino]-benzoic acid methyl ester (3.72 g) in MeOH (250 mL) was added a 1 N NaOH solution (12.2 ml, 2 eq.). The mixture was stirred to reflux overnight and then the solvent was evaporated off. The residue was treated with 1 N NH₄Cl solution and extracted with AcOEt. The organic layer was dried over Na₂SO₄, filtered, evaporated to dryness. The resulting powder was washed with hot MeOH to afford the title compound as an orange powder (2.88 g), which contains the 2-trityl isomer as a minor component.

Calcd. Mass for C₃₉H₂₉N₅O₂ (M+1)⁺: 600.2400. Found (H.R.M.S): 600.2379

Intermediate G:

4-[4-(3-Pyridin-2-yl-1-trityl-1H-pyrazol-4-yl)-pyridin-2-ylamino]-phenol

To a suspension of 2-bromo-4-(3-pyridin-2-yl-1-trityl-1H-pyrazol-4-yl)pyridine (4.8 g) in toluene (100 ml) were added Pd₂(dba)₃ (165 mg, 2 mol %), binap (222 mg, 4 mol %), 4-Benzyloxy-phenylamine hydrochloride (3 g, 1.4 eq) and sodium tert-butoxide (2.4 g, 2.8 eq). The reaction mixture was stirred at 80° C. overnight. The mixture was poured in cold water and extracted with toluene. The organic layer was washed with a saturated NaCl solution, dried over Na₂SO₄, filtered and evaporated to dryness. The resulting solid was recrystallised from EtOH to afford the title compound (5.7 g, 97%) which contains the 2-trityl isomer as a minor component.

The mixture of the two isomers (5.7 g) was dissolved in EtOH/THF (250 ml) and Pd/C,10% (500 mg) was added. The reaction was stirred under an atmospheric pressure of hydrogen at 40° C. Overnight. The mixture was filtered through celite. The filtrate was evaporated under reduced pressure to afford the title compound, and the 2-trityl isomer as a minor component, as a brown solid after precipitation in iPr₂O(4.8 g, 97%).

m.p:198° C.

### EXAMPLES

### Example 1

(4-Chloro-phenyl)-[4-(3-pyridin-2-yl)-1H-pyrazol-4-yl)-pyridin-2-yl]-amine

To a suspension of Intermediate D1 (3.15 g) in toluene (60 ml) were added Pd₂(dba)₃ (106 mg, 0.02 mol %), binap (144 mg, 0.04 mol %), 4-chloroaniline (890 mg, 1.2 eq) and sodium tert-butoxide (780 mg, 1.4 eq). The reaction mixture was stirred at 90° C. overnight. The mixture was washed with diethyl dithiocarbamic acid, sodium salt trihydrate (1M) and extracted with toluene. The organic layer was washed with water, dried over Na₂SO₄, filtered, evaporated to dryness and chromatographed [CH₂Cl₂ then CH₂Cl₂/MeOH 99:1] to afford 2.8 g (white solid) of 1-trityl isomer+2-trityl isomer as a minor component.

The mixture of the two isomers was treated with HCl (1N)/MeOH (2/3, 100 ml) and heated at reflux for 18 h. The solvent was removed under reduce pressure and the resulting residue taken up into water and washed with CH₂Cl₂. The aqueous layer was basified with NaOH (1N, 50 ml) and extracted with ethyl acetate. The organic layer was dried over Na₂SO₄, filtered, and evaporated to dryness. The resulting solid was recrystallised from iPrOH to afford the title compound (745 mg) as an off-white solid.

m.p :216° C.

MS m/z 348 (MH+)

### Example 2

Phenyl-[4-(3-pyridin-2-yl)-1H-pyrazol-4-yl)-pyridin-2-yl]-amine

A procedure similar to that for Example 1 using aniline (123 mg) gave, after recrystallisation from iPrOH, the title compound as white crystals (210 mg).

m.p:230° C.

GC-MS. 312

### Example 3

(4-Fluoro-phenyl)-[4-(3-pyridin-2-yl)-1H-pyrazol-4-yl)-pyridin-2-yl]-amine

A procedure similar to that for Example 1 using 4-fluoroaniline (444 mg) gave, after recrystallisation from iPrOH, the title compound as white crystals (380 mg).

m.p :200° C.

MS m/z 332 (MH+)

### Example 4

Furan-2-ylmethyl-[4-(3-pyridin-2-yl)-1H-pyrazol-4-yl)-pyridin-2-yl]-amine

A procedure similar to that for Example 1 using furfurylamine (388 mg) gave, after recrystallisation from EtOH, the title compound as white crystals (70 mg).

m.p :200° C.

MS m/z 318 (MH+)

### Example 5

(3-Methanesulfonyl-phenyl)-[4-(3-pyridin-2-yl)-1H-pyrazol-4-yl)-pyridin-2-yl]-amine

A procedure similar to that for Example 1 using 3-methylsulphonylaniline, hydrochloride (500 mg) gave, after recrystallisation with iPrOH/iPr₂O, the title compound as a white solid (200 mg, 27.6%).

m.p :146° C.

MS m/z 392 (MH+)

### Example 6

3-[4-(3-Pyridin-2-yl)-1H-pyrazol-4-yl)-pyridin-2-yl-amino]-benzonitrile

A procedure similar to that for Example 1 using 3-amino-benzonitrile (480 mg) gave, after recrystallisation from iPrOH, the title compound as white crystals (515 mg, 42%).

m.p :228° C.

MS m/z 339 (MH+)

### Example 7

2-Methoxy-4-[4-(3-pyridin-2-yl)-1H-pyrazol-4-yl)-pyridin-2-yl-amino]-benzonitrile

A procedure similar to that for Example 1 using 4-amino-2-methoxy-benzonitrile (518 mg) gave, after recrystallisation from MeOH, the title compound as yellow crystals (260 mg).

m.p :280° C.

MS m/z 369 (MH+)

### Example 8

[4-(3-Pyridin-2-yl)-1H-pyrazol-4-yl)-pyridin-2-yl]-[3-(2-piperidin-1-yl-ethoxy)-phenyl]-amine

A procedure similar to that for Example 1 using 3-(2-piperidinoethoxy)aniline (1 g) gave, after recrystallisation from iPrOH, the title compound as a white powder (233 mg, 16%).

m.p:115° C.

MS m/z 441 (MH+)

### Example 9

[4-(3-Pyridin-2-yl)-1H-Pyrazol-4-yl)-pyridin-2-yl]-[3-(2-pyrrolidin-1-yl-ethoxy)-phenyl]-amine

A procedure similar to that for Example 1 using 3-(2-pyrrolidinoethoxy)aniline (18.7 g) gave the title compound as an off-white powder (14.65 g, 49%).

m.p :141° C.

MS m/z 427 (MH+)

### Example 10

(3-Chloro-phenyl)-[4-(3-pyridin-2-yl)-1H-pyrazol-4-yl)-pyridin-2-yl]-amine

A procedure similar to that for Example 1 using 3-chloroaniline (800 mg) gave the title compound as a pale pink solid. (430 mg, 49%).

m.p:205° C.

MS m/z 348 (MH+)

### Example 11

[4-(3-Pyridin-2-yl-1H-pyrazol-4-yl)-pyridin-2-yl]-m-tolyl-amine

A procedure similar to that for Example 1 using m-tolylamine (417 mg) gave, after recrystallisation from iPrOH, the title compound as a white powder (550 mg, 56%).

m.p :194° C.

MS m/z 328 (MH+)

### Example 12

[4-(3-Pyridin-2-yl-1H-pyrazol-4-yl)-pyridin-2-yl]-[4-(2-pyrrolidin-1-yl-ethoxy)-phenyl]-amine

A procedure similar to that for Example 1 using 4-(2-pyrrolidin-1-yl-ethoxy)-phenylamine (500 mg) gave, after recrystallisation from MeOH, the title compound as white crystals (310 mg, 48%).

m.p :224° C.

MS m/z 427 (MH+)

### Example 13

(3,4-Dimethoxy-phenyl)-[4-(3-pyridin-2-yl-1H-pyrazol-4-yl)-pyridin-2-yl]-amine

A procedure similar to that for Example 1 using 3,4-dimethoxy-phenylamine (642 mg) gave, after recrystallisation from iPrOH, the title compound as a white crystals (480 mg, 40.3%).

m.p:210° C.

MS m/z 374 (MH+)

### Example 14

(4-Chloro-3-methyl-phenyl)-[4-(3-pyridin-2-yl-1H-pyrazol-4-yl)-pyridin-2-yl]-amine

A procedure similar to that for Example 1 using 4-chloro-3-methyl-phenylamine (500 mg) gave, after recrystallisation from iPrOH, the title compound as white crystals (470 mg, 52%).

m.p :212° C.

MS m/z 363 (MH+)

### Example 15

2-Chloro-5-[4-(3-pyridin-2-yl-1H-pyrazol-4-yl)-pyridin-2-ylamino]-benzonitrile

A procedure similar to that for Example 1 using 5-amino-2-chloro-benzonitrile (532 mg) gave, after recrystallisation from MeOH, the title compound as white crystals (700 mg, 75.2%).

m.p :250° C.

MS m/z 374 (MH+)

### Example 16

4-[4-(3-Pyridin-2-yl-1H-pyrazol-4-yl)-pyridin-2-ylamino]-phenol

Intermediate G (400 mg) was treated with HCl(1N)/MeOH (2/3, 25 ml) and heated at reflux overnight. The solvent was removed under reduced pressure and the resulting residue taken up into water and washed with CH₂Cl₂. The aqueous layer was basified with NaHCO₃ to pH>10 and the precipitate was filtered off and washed with water. The resulting solid was recrystallised from MeOH to afford the title compound (160 mg, 69%) as white crystals.

m.p :268° C.

MS m/z 328 (M−1)

### Example 17

(4-Methoxy-phenyl)-[4-(3-pyridin-2-yl-1H-pyrazol-4-yl)-pyridin-2-yl]-amine

A procedure similar to that for Example 1 using 4-methoxyphenylamine (86.2 mg) gave, after recrystallisation from EtOH, the title compound (40 mg, 20%) as colourless crystals.

m.p :219° C.

MS m/z 344 (MH+)

### Example 18

[4-(3-Pyridin-2-yl-1H-pyrazol-4-yl)-pyridin-2-yl]-p-tolyl-amine

A procedure similar to that for Example 1 using p-tolylamine (148 mg) gave, after chromatography [CH₂Cl₂/MeOH 95:5], the title compound (90 mg, 30%) as colourless crystals.

m.p :221° C.

MS m/z 328 (MH+)

### Example 19

[4-(3-Pyridin-2-yl-1H-pyrazol-4-yl)-pyridin-2-yl]-(3-trifluoromethoxy-phenyl)-amine

A procedure similar to that for Example 1 using 3-trifluoromethoxy-phenylamine (424 mg) gave, after recrystallisation from CH₂Cl₂, the title compound (180 mg, 24.6%) as a white solid.

m.p :102.8° C.

MS m/z 398 (MH+)

### Example 20

(3,4-Dimethyl-phenyl)-[4-(3-pyridin-2-yl-1H-pyrazol-4-yl)-pyridin-2-yl]-amine

A procedure similar to that for Example 1 using 3,4-dimethyl-phenylamine (598.11 mg) gave, after recrystallisation from MeOH, the title compound (310 mg, 25.8%) a a white solid.

m.p :220° C.

MS m/z 342 (MH+)

### Example 21

[4-(3-Pyridin-2-yl-1H-pyrazol-4-yl)-pyridin-2-yl]-(4-trifluoromethyl-phenyl)-amine

A procedure similar to that for Example 1 using 4-(trifluoromethyl)aniline (90 μl, 1.2 eq) gave the title compound (60 mg, 27%) as a yellow powder.

m.p :207° C.

MS m/z 382 (MH+)

### Example 22

(3-Isopropoxy-phenyl)-[4-(3-pyridin-2-yl-1H-pyrazol-4-yl)-pyridin-2-yl]-amine

A procedure similar to that for Example 1 using 3-isopropoxy-phenylamine (290 mg, 1.3 eq) gave after chromatography [CH₂Cl₂/MeOH 90:10], the title compound as a yellow powder (30 mg, 12%).

m.p: 218° C.

MS m/z 372 (MH+)

### Example 23

(4-Methanesulfonyl-phenyl)-[4-(3-pyridin-2-yl-1H-pyrazol-4-yl)-pyridin-2-yl]-amine

A procedure similar to that for Example 1 using 4-methanesulfonyl-phenylamine (416 mg, 1.2 eq) gave, after recrystallisation from iPrOH, the title compound as a yellow powder (207 mg, 26%).

m.p: 134° C.

MS m/z 392 (MH+)

### Example 24

2-Methoxy-4-{4-[3-(6-methyl-pyridin-2-yl)-1H-pyrazol-4-yl]-pyridin-2-ylamino}-benzonitrile

A procedure similar to that for Example 1 using Intermediate D2 (3.19 g) and 4-amino-2-methoxy-benzonitrile (1.2 g) gave the title compound (1.3 g, 59.4%) as an off-white solid.

m.p :125° C.

MS m/z 383(MH+)

### Example 25

Morpholin-4-yl-{4-[4-(3-pyridin-2-yl-1H-pyrazol-4-yl)-pyridin-2-ylamino]-phenyl}methanone

A solution of intermediate F (1 g) in DMF was treated with HOBT (293 mg, 1.3 eq.), EDCl (401 mg, 1.3 eq.), DIEA (365 μL, 1.3 eq.) and morpholine (185 μL, 1.3 eq.). The resulting mixture was stirred for 48 hours at r.t. The solvent was evaporated off and the residue was washed with water and extracted with CH₂Cl₂. The organic layer was dried over Na₂SO₄ and evaporated to afford the title compound (1.1 g) as a colourless oil, which contains the 2-trityl isomer as a minor component . MS m/z 669 (MH+). The mixture of the two isomers (1.1 g) was treated with HCl (1N)/MeOH (2/3, 100 ml) and heated at reflux overnight. The solvent was removed under reduced pressure and the resulting residue taken up into 1N NaOH solution and extracted with ethyl acetate. The organic layer was dried over Na₂SO₄, filtered, and evaporated to dryness. The resulting product was chromatographed [CH₂Cl₂/MeOH 95:5] and recrystallised from iPr₂O to afford the title compound (116 mg, 16%) as an off-white powder.

m.p :240° C.

Calcd. Mass for C₂₄H₂₂N₆O₂: (M+1)⁺:427.1882. Found (H.R.M.S): 427.1892.

### Example 26

(4-Methyl-piperazin-1-yl)-{4-[4-(3-pyridin-2-yl-1H-pyrazol-4-yl)-pyridin-2-ylamino]-phenyl}-methanone

A procedure similar to that for Example 25 using Intermediate F (420 mg) and N-Methyl-piperazine (100 mg, 1.4 eq.) gave after recrystallisation from AcOEt, the title compound as white crystals (94 mg, 30.6%).

m.p: 200° C.

MS m/z 440 (MH+)

### Example 27

[4-(2-Piperidin-1-yl-ethoxy)phenyl]-[4-(3-pyridin-2-yl-1H-pyrazol-4-yl)-pyridin-2-yl]-amine

To a solution of intermediate G (400 mg) in acetone (30 mL) was added Cs₂CO₃ (1.17 g, 5.14 eq.) and 1-(2-chloroethyl)-piperidine hydrochloride (147 mg, 1.14 eq.). The mixture was stirred to reflux for 48 hours and filtrated off. The filtrate was evaporated off and the residue was diluted in CH₂Cl₂ and washed with water. The organic layer was dried over Na₂SO₄, filtered and evaporated to dryness. The mixture of 1-trityl and 2-trityl isomers was treated with HCl (1N)/MeOH (2/3, 30 ml) and heated at reflux overnight. The solvent was removed under reduced pressure and the resulting residue taken up into water and washed with CH₂Cl₂. The aqueous layer was basified to pH14 with NaOH (1N) and extracted with CH₂Cl₂. The organic layer was dried over Na₂SO₄, filtered, and evaporated to dryness. The resulting solid was recrystallised from iPrOH to afford the title compound (150 mg, 48.7%) as white crystals.

m.p :176° C.

MS m/z 441 (MH+)

### Example 28

[3-(1-Methyl-1H-imidazol-2-ylmethoxy)-phenyl]-[4-(3-pyridin-2-yl-1H-pyrazol-4-yl)-pyridin-2-yl]-amine

A procedure similar to that for Example 27 using 2-chloromethyl-1-methyl-1H-imidazole (190 mg) gave, after chromatography [CH₂Cl₂/MeOH 95:5 then CH₂Cl₂/MeOH 90:10], the title compound (80 mg, 21.7%) an off-white solid.

m.p: 120° C.

MS m/z 424 (MH+)

Assays

Assay 1

The potential for compounds of the invention to inhibit TGF-β signaling may be demonstrated, for example, using the following in vitro assay.

The assay was performed in HepG2 cells stably transfected with the PAI-1 promoter (known to be a strong TGF-β responsive promoter) linked to a luciferase (firefly) reporter gene. The compounds were selected on their ability to inhibit luciferase activity after exposure to TGF-β. In addition cells were transfected with a second luciferase (Renilla) gene which was not driven by a TGF-β responsive promoter and was used as a toxicity control.

(96 well-)microplates are seeded, using a multidrop apparatus, with the stably transfected cell line at a concentration of 35000 cells per well in 200 μl of serum-containing medium. These plates are placed in a cell incubator.

18 to 24 hours later (Day 2), cell-incubation procedure is launched. Cells are incubated with TGF-β and a candidate compound at concentrations in the range 50 nM to 10 μM (final concentration of DMSO 1%). The final concentration of TGF-β (rhTGFβ-1) used in the test is 1 ng/mL. Cells are incubated with a candidate compound 15-30 mins prior to the addition of TGF-β. The final volume of the test reaction is 150 μl. Each well contains only one candidate compound and its effect on the PAI-1 promoter is monitored.

Columns 11 and 12 are employed as controls. Column 11 contains 8 wells in which the cells are incubated in the presence of TGF-β, without a candidate compound. Column 11 is used to determine the ‘reference TGF-β induced firefly luciferase value’ against which values measured in the test wells (to quantify inhibitory activity) may be compared. In wells A12 to D12, cells are grown in medium without TGF-β. The firefly luciferase values obtained from these positions are representive of the ‘basal firefly luciferase activity’. In wells E12 to H12, cells are incubated in the presence of TGF-β and 500 μM CPO (Cyclopentenone, Sigma), a cell toxic compound. The toxicity is revealed by decreased firefly and renilla luciferase activities (around 50% of those obtained in column 11).

12 to 18 hours later (day 3), the luciferase quantification procedure is launched. The following reactions are performed using reagents obtained from a Dual Luciferase Assay Kit (Promega). Cells are washed and lysed with the addition of 10 μl of passive lysis buffer (Promega). Following agitation (15 to 30 mins), luciferase activities of the plates are read in a dual-injector luminometer (BMG lumistar). For this purpose, 50 μl of luciferase assay reagent and 50 μl of ‘Stop & Glo’ buffer are injected sequentially to quantify the activities of both luciferases. Data obtained from the measurements are processed and analysed using suitable software. The mean Luciferase activity value obtained in wells A11 to H11 (Column 11, TGF-β only) is considered to represent 100% and values obtained in wells A12 to D12 (cells in medium alone) give a basal level (0%). For each of the compounds tested, a concentration response curve is constructed from which an IC₅₀ value can be determined graphically.

Assay 2

The potential for compounds of the invention to inhibit the kinase Alk5 receptor may be demonstrated, for example, using the following in vitro assay.

The kinase domain of Alk5 was cloned and expressed in a baculovirus/Sf9 cells system. The protein (amino acids 162 to 503) was 6-His tagged in C-terminus. After purification by affinity chromatography using a Ni²⁺ column, the autophosphorylation was tested. The enzyme was incubated in a medium containing: Tris 50 mM pH 7.4; NaCl 100 mM; MgCl₂ 5 mM ; MnCl₂ 5 mM ; DTT 10 mM. The enzyme was preincubated with the compounds (0.1% DMSO final in the test) 10 minutes at 37° C. The reaction was initialised by the addition of 3 μM ATP (0.5 μCi gamma-33P-ATP). After 15 minutes at 37° C. the reaction was stopped by addition of SDS-PAGE sample buffer (50 mM Tris-HCl, pH 6.9, 2.5% glycerol, 1% SDS, 5% beta-mercaptoethanol). The samples were boiled for 5 minutes at 95° C. and run on a 12% SDS-PAGE. The dried gels were exposed to a phosphor screen over-night. Alk5 autophosphorylation was quantified using a STORM (Molecular Dynamics).

Biological Data

The compounds of Examples 1-28 were tested in vitro, using the biological assays described above. All of the compounds had an IC₅₀ value of 5 μM or below in Assay 1, and an IC₅₀ value of 1 μM or below in Assay 2.

1. 1. A compound of formula (I), 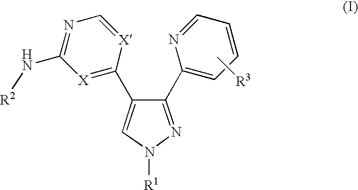
wherein,
R¹ is selected from H, C₁₋₄ alkyl or CH₂CONR⁴R⁵, wherein R⁴ is selected from H or C₁₋₄ alkyl and R⁵ is C₁₋₄ alkyl;
R² is selected from —(CH₂)ₙ-phenyl, —(CH₂)ₙ-heterocyclyl, —(CH₂)ₙ-heteroaryl, each of which may be further substituted by one or more substituents, which may be the same or different, selected from halo (such as fluoro, chloro, bromo), —CN, —CF₃, —OH, —OCF₃, C₁₋₄ alkyl or C₁₋₄ alkoxy, —NO₂, —NH₂, —NR⁴R⁶, —CONR⁴R⁶, —NHCOR⁴, —SO₂R⁴, —SO₂NHR⁴, —O(CH₂)ₙNR⁴R⁶;
R³ is selected from H, halo, —CN, —CF₃, C₁₋₄ alkyl or C₁₋₄ alkoxy;
R⁴ is selected from H or C₁₋₄ alkyl;
R⁵ is C₁₋₄ alkyl;
R⁶ is selected from heterocyclyl or heteroaryl;
or R⁴R⁶ together with the nitrogen atom to which they are attached form a 3, 4, 5, 6 or 7 membered saturated or unsaturated ring which may additionally contain one or more heteroatoms selected from N, S or O, and wherein the ring may be further substituted by one or more substitutents selected from halo (such as fluoro, chloro, bromo), —CN, —CF₃, —OH, —OCF₃, C₁₋₄ alkyl and C₁₋₄ alkoxy;
n is 0, 1, 2, 3, 4 or 5;
X and X′, which may be the same or different, are each selected from CH or N, provided that X and X′ are not both N,
and salts and solvates thereof.

2. A compound of formula (I) as claimed in claim 1, wherein R¹ is H or C₁₋₄ alkyl.

3. A compound of formula (I) as claimed in claim 1 or claim 2, wherein R¹ is H.

4. A compound of formula (I) as claimed in any one of claims 1 to 3 wherein n is 0 or 1.

5. A compound of formula (I) as claimed in any one of claims 1 to 4, wherein R³ is located at the C(3) or C(6) position of the pyridine ring.

6. A compound of formula (I) as claimed in any one of claims 1 to 5, wherein R³ is H or C₁₋₄ alkyl.

7. A compound of formula (I) as claimed in any of the preceding claims, wherein R⁴ and R⁶ together with the nitrogen atom to which they are attached represent pyrroline, pyrrolidine, imidazole, N-substituted imidazole, imidazoline, imidazolidine, pyrazole, pyrazoline, pyrazolidine, piperidine, morpholine, thiomorpholine, piperazine, N-substituted piperazine.

8. A compound of formula (I) as claimed in any one of the preceding claims, wherein R⁴ and R⁶ together with the nitrogen atom to which they are attached represent pyrrolidine, piperidine, morpholine, piperazine, N-substituted piperazine, imidazole, or N-substituted imidazole.

9. A compound of formula (I) as claimed in claim 1 selected from:
(4-Chlorophenyl)-[4-(3-pyridin-2-yl)-1H-pyrazol-4-yl)-pyridin-2-yl]-amine;
Phenyl-[4-(3-pyridin-2-yl)-1H-pyrazol-4-yl)-pyridin-2-yl]-amine;
(4-Fluoro-phenyl)-[4-(3-pyridin-2-yl)-1H-pyrazol-4-yl)-pyridin-2-yl]-amine;
Furan-2-ylmethyl-[4-(3-pyridin-2-yl)-1H-pyrazol-4-yl)-pyridin-2-yl]-amine;
(3-Methanesulfonyl-phenyl)-[4-(3-pyridin-2-yl)-1H-pyrazol-4-yl)-pyridin-2-yl]-amine;
3-[4-(3-Pyridin-2-yl)-1H-pyrazol-4-yl)-pyridin-2-yl-amino]-benzonitrile;
2-Methoxy-4-[4-(3-pyridin-2-yl)-1H-pyrazol-4-yl)-pyridin-2-yl-amino]-benzonitrile;
[4-(3-Pyridin-2-yl)-1H-pyrazol-4-yl)-pyridin-2-yl]-[3-(2-piperidin-1-yl-ethoxy)-phenyl]-amine; and
[4-(3-Pyridin-2-yl)-1H-pyrazol-4-yl)-pyridin-2-yl]-[3-(2-pyrrolidin-1-yl-ethoxy)-phenyl]-amine;
(3-Chloro-phenyl)-[4-(3-pyridin-2-yl)-1H-pyrazol-4-yl)-pyridin-2-yl]-amine;
[4-(3-Pyridin-2-yl-1H-pyrazol-4-yl)-pyridin-2-yl]-m-tolyl-amine;
[4-(3-Pyridin-2-yl-1H-pyrazol-4-yl)-pyridin-2-yl]-[4-(2-pyrrolidin-1-yl-ethoxy)-phenyl]-amine;
(3,4-Dimethoxy-phenyl)-[4-(3-pyridin-2-yl-1H-pyrazol-4-yl)-pyridin-2-yl]-amine;
(4-Chloro-3-methyl-phenyl)-[4-(3-pyridin-2-yl-1H-pyrazol-4-yl)-pyridin-2-yl]-amine;
2-Chloro-5-[4-(3-pyridin-2-yl-1H-pyrazol-4-yl)-pyridin-2-ylamino]-benzonitrile;
4-[4-(3-Pyridin-2-yl-1H-pyrazol-4-yl)-pyridin-2-ylamino]-phenol;
(4-Methoxy-phenyl)-[4-(3-pyridin-2-yl-1H-pyrazol-4-yl)-pyridin-2-yl]-amine;
[4-(3-Pyridin-2-yl-1H-pyrazol-4-yl)-pyridin-2-yl]-p-tolyl-amine;
[4-(3-Pyridin-2-yl-1H-pyrazol-4-yl)-pyridin-2-yl]-(3-trifluoromethoxy-phenyl)-amine;
(3,4-Dimethyl-phenyl)-[4-(3-pyridin-2-yl-1H-pyrazol-4-yl)-pyridin-2-yl]-amine;
[4-(3-Pyridin-2-yl-1H-pyrazol-4-yl)-pyridin-2-yl]-(4-trifluoromethyl-phenyl)-amine;
(3-Isopropoxy-phenyl)-[4-(3-pyridin-2-yl-1H-pyrazol-4-yl)-pyridin-2-yl]-amine;
(4-Methanesulfonyl-phenyl)-[4-(3-pyridin-2-yl-1H-pyrazol-4-yl)-pyridin-2-yl]-amine;
2-Methoxy-4-{4-[3-(6-methyl-pyridin-2-yl)-1H-pyrazol-4-yl]-pyridin-2-ylamino}-benzonitrile;
Morpholin-4-yl-{4-[4-(3-Pyridin-2-yl-1H-pyrazol-4-yl)-pyridin-2-ylamino]-phenyl}methanone;
(4-Methyl-piperazin-1-yl)-{4-[4-(3-pyridin-2-yl-1H-pyrazol-4-yl)-pyridin-2-ylamino]-phenyl}-methanone;
[4-(2-Piperidin-1-yl-ethoxy)-phenyl]-[4-(3-pyridin-2-yl-1H-pyrazol-4-yl)-pyridin-2-yl]-amine; and
[3-(1-Methyl-1H-imidazol-2-ylmethoxy)-phenyl]-[4-(3-pyridin-2-yl-1H-pyrazol-4-yl)-pyridin-2-yl]-amine;
and salts and solvates thereof.

10. A pharmaceutical composition comprising a compound of formula (I) as claimed in claim any one of claims 1 to 9, together with a pharmaceutically acceptable diluent or carrier.

11. A compound of formula (I) as claimed in any one of claims 1 to 9, for use as a medicament.

12. The use of a compound of formula (I) as claimed in any one of claims 1 to 9, for the manufacture of a medicament for the treatment and/or prophylaxis of a disorder characterised by the overexpression of TGF-β.

13. A method for the treatment of a human or animal subject with a disorder characterised by the overexpression of TGF-β, which method comprises administering to said human or animal subject an effective amount of a compound of formula (I) as claimed in any one of claims 1 to 9 or a physiologically acceptable salt or solvate thereof.

14. A process for the preparation of a compound of formula (I) as claimed in any one of claims 1 to 9, comprising:
a) treatment of a compound of formula (D) with an amine R²NH₂ in the presence of Pd₂(dba)₃, and binap, 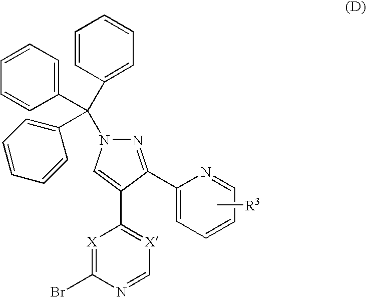
wherein R³ is defined above; and
b) subsequent removal of the protecting group from the resulting product.

15. A process for the preparation of a compound of formula (I) as claimed in any one of claims 1 to 9, comprising:
N-alkylation of a compound of formula (I) wherein R¹ is H, in the presence of a suitable base.
